(19) United States
(12) Patent Application Publication
(10) Pub. No.: US 2011/0209707 A1
(43) Pub. Date: Sep. 1, 2011

(54) Method And Apparatus For Oxygen Reprocessing Of Expiratory Gases In Mechanical Ventilation

(75) Inventor: Gregory L. Terhark, Centennial, CO (US)

(73) Assignee: Nellcor Puritan Bennett LLC, Boulder, CO (US)

(21) Appl. No.: 12/714,221

(22) Filed: Feb. 26, 2010

(51) Int. Cl. A61M 16/12; A61M 16/22; G06F 3/048

(52) U.S. Cl. 128/205.11; 128/205.12; 715/771

(57) ABSTRACT

This disclosure describes systems and methods for oxygen reprocessing of respiratory gases in mechanical ventilation. Embodiments described herein provide methods for oxygen reprocessing of expiratory gases wherein exhaled air is reprocessed using filters and adsorbers and delivered to a patient based on a clinically specified oxygen concentration. Embodiments described herein provide for graphical display of the oxygen concentration of the product gas of oxygen reprocessing. Embodiments described herein also disclose an automated ventilator functionality whereby oxygen concentration may be set by clinicians through a user interface display, or a “Oxygen Reprocessing Input” screen, from a general ventilation input screen. Embodiments described herein also disclose a ventilator system configured to undergo oxygen reprocessing of expiratory gases and to provide the product gas to the patient for inspiration.

### INTRODUCTION

A ventilator is a device that mechanically helps patients breathe by replacing some or all of the muscular effort required to inflate and deflate the lungs. Ventilatory assistance is indicated for certain diseases affecting the musculature required for breathing, such as muscular dystrophies, polio, amyotrophic lateral sclerosis (ALS), and Guillain-Barré syndrome. Mechanical ventilation may also be required during the sedation associated with surgery and as the result of various injuries, such as high spinal cord injuries and head traumas.

Ventilators may provide assistance according to a variety of methods based on the needs of the patient. These methods include volume-cycled and pressure-cycled methods. Specifically, volume-cycled methods may include among others, Pressure-Regulated Volume Control (PRVC), Volume Ventilation (VV), and Volume Controlled Continuous Mandatory Ventilation (VC-CMV) techniques. Pressure-cycled methods may involve, among others, Assist Control (AC), Synchronized Intermittent Mandatory Ventilation (SIMV), Controlled Mechanical Ventilation (CMV), Pressure Support Ventilation (PSV), Continuous Positive Airway Pressure (CPAP), or Positive End Expiratory Pressure (PEEP) techniques.

A ventilator can also be used to provide inspiratory air with highly concentrated oxygen content to a patient. Ventilators dispense between 21-100% oxygen concentrations for clinical use. With a normal 4% reduction (21%-17%) in oxygen concentration between room air and exhaled room air in normal, resting subjects, the concentration of oxygen in the exhaled gases of even the most acutely ill would contain small to significant amounts of unused oxygen.

The use of continuous flows in ventilators to reduce the work of breathing also directly dispenses significant amounts of oxygen during ventilation. As described herein below, reprocessing air exhaled by a patient during ventilation for inspiration would result in significant cost savings of providing compressed and/or liquid oxygen, as well as simpler transport of patients on the ventilator.

### Method and System for Oxygen Reprocessing of Expiratory Gases in Mechanical Ventilation

This disclosure describes methods and apparatus for oxygen reprocessing of expiratory gases in a ventilator. As used in this disclosure, oxygen reprocessing is the process of filtering and concentrating gases expired by a patient and providing these concentrated expiratory gases to the patient for inspiration. The present disclosure may be applicable for adult, pediatric, and neonatal ventilatory techniques, as required by the diverse care plans of various patients.

The filtration process used in oxygen reprocessing could utilize any number of bacterial and/or viral filters. Bacterial and viral filters provide protection against different types of particles including bacteria, viruses, and water droplets. The exhaled air from a patient can be filtered during the flow of the ventilation process creating filtered exhaled air. Furthermore, filters can be designed to provide a low and stable breathing resistance. This low breathing resistance can be maintained even when the filter is wet resulting in minimal disruption to the continuous flow of the ventilator.

The concentration process used in oxygen reprocessing can be accomplished by providing the filtered expired air to one or more adsorbers. Adsorption is a process used to separate a gas species from a mixture of gases. The result of the process is product gas more highly concentrated in the remaining molecules of the gas mixture.

An adsorber is typically comprised of one or more adsorbent beds. Each adsorbent bed can contain a bed of molecules. The nature of the bed of molecules in the adsorbent bed is determined by the molecular characteristics and affinities of the gas species. Separation of the gas species from the gas mixture is accomplished when the species adheres to the bed of molecules. Alternatively, an adsorbent bed can be comprised of a molecular sieve. A molecular sieve is a material containing tiny pores of precise and uniform sizes. The size of the pores depends on the size of the molecules in the species gas. The pores need to be large enough to allow molecules of the species gas to diffuse through but small enough to filter out the remaining molecules in the gas mixture. Molecular sieves can consist of aluminosilicate minerals, clays, porous glasses, microporous charcoals, zeolites, active carbons, or synthetic compounds with open structures through which molecules can pass.

Adsorption typically utilizes one or both of two technologies—temperature swing adsorption (TSA) and pressure swing adsorption (PSA). In both of these technologies, the adsorbent bed is exposed to the feed gas mixture for a fixed period of time so that the adsorbent can adsorb the species gas. The fixed period of exposure time is sufficiently brief so that the species gas does not “break-through” and rejoin the feed gas mixture. The concentrated feed gas mixture exits the adsorbent bed as product gas. During desorption, the flow feed air is shut off and the adsorbent is exposed to a flow of regeneration gas that strips the species gas from the adsorbent. In TSA, the adsorbents are exposed to a flow of heated regeneration gas. In PSA, the adsorbents are exposed to a regeneration gas with lower pressure than the feed gas. This regeneration gas is then exhausted from the adsorbent bed along with the adsorbed species gas and other impurities.

Adsorption can also utilize dual molecular sieves during the adsorption and desorption cycles. Particularly, when one molecular sieve is adsorbing the other molecular sieve is desorbing. These adsorption/desorption cycles can be regulated by the TSA mechanisms, PSA mechanisms or by other suitable regulation devices, such as solenoid actuators.

Oxygen concentration is typically regulated by measuring the oxygen content of the product gas. Many different methods exist to measure the concentration of oxygen in a gas. Among the most commonly used are electrochemical sensors, partial pressure sensors, zirconia sensors, and paramagnetic measurement. An oxygen concentrator operating at optimum efficiency typically generates air with an oxygen concentration of 94%. The accepted lower threshold for oxygen concentration units is typically an 85% oxygen concentration in the output concentrated air but can be any percentage.

During ventilation, the molecular content of inspiratory air received by a patient through ventilation is regulated by a clinician. A clinician will often specify a desired FIO₂ level for inspiration. FIO₂ is the fractional oxygen concentration in inspired air. Clinicians will often set a higher FIO₂ concentration for patients who are having difficulty absorbing oxygen into their bloodstreams. Once a clinician specifies a FIO₂ level at the ventilator, the ventilator uses a blending algorithm to produce the desired concentration. A blending algorithm is used to mix the air provided for inspiration with room/air and or pure oxygen. The concentration of the blended air reflects the FIO₂ level specified by the clinician.

Embodiments herein describe a method executable on a computerized ventilator for oxygen reprocessing of expiratory gases during ventilation of a patient. The method utilizes both a carbon dioxide adsorber and an argon/nitrogen adsorber to increase oxygen concentration in the product gas. Embodiments herein also describe a reprocessing module for oxygen reprocessing of expiratory gases during ventilation of a patient, comprising a carbon dioxide adsorber, an argon/nitrogen adsorber, and a threshold testing module. Embodiments herein also describe a graphical user interface for configuring oxygen reprocessing of expiratory gases on a ventilator, the ventilator including a computer having a user interface including the graphical user interface for inputting parameters.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 is a diagram illustrating a representative ventilator system utilizing an endotracheal tube for air delivery to a patient's lungs.

FIG. 2 is a block-diagram illustrating an embodiment of a ventilatory system for monitoring oxygen concentration and molecular sieve duration of use in the reprocessing module.

FIG. 3 is a block-diagram illustrating an embodiment of a reprocessing module for oxygen reprocessing of expiratory gases as described herein.

FIG. 4 is a flow-diagram illustrating an embodiment of a method for oxygen reprocessing of expiratory gases in a ventilator.

FIG. 5 is a block diagram of a graphical user interface illustrating an embodiment of an oxygen reprocessing input screen.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other systems in which gas volume, pressure, and flow should be carefully regulated.

This disclosure describes methods and apparatus for oxygen reprocessing of expiratory gases in a ventilator. As described above, oxygen reprocessing involves both filtration and concentration of expiratory gasses in a ventilator. Reprocessed oxygen may result in significant cost savings by allowing patients to reuse their expired gasses instead of providing them with compressed and/or liquid oxygen. Using reprocessed oxygen instead of these external oxygen sources will also result in simpler transport of patients on a ventilator.

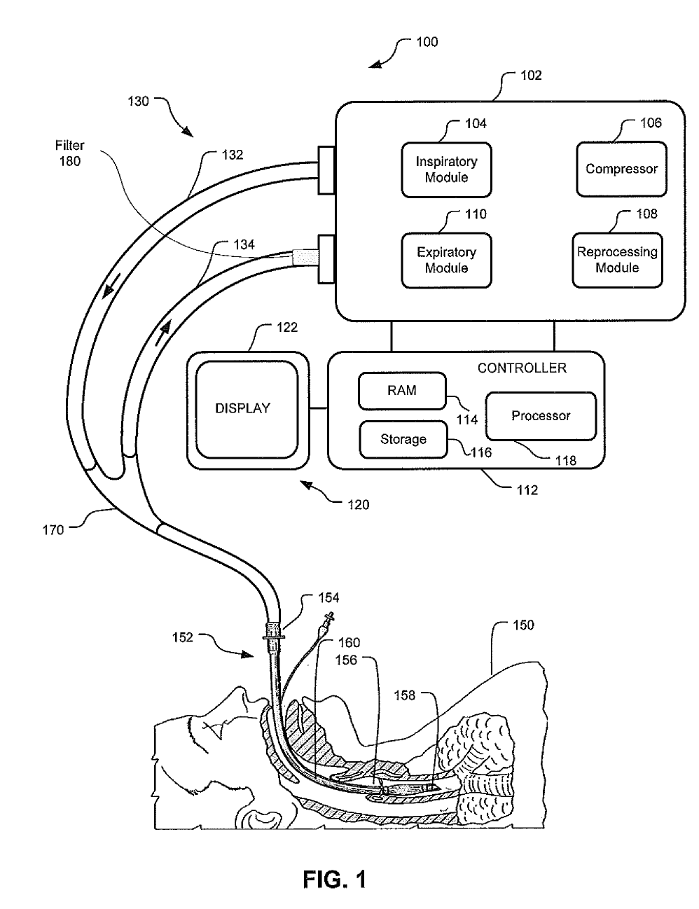

FIG. 1 illustrates an embodiment of a ventilator 100 connected to a human patient 150. Ventilator 100 includes a pneumatic system 102 for circulating breathing gases to and from patient 150 via the ventilation tubing system 130, which couples the patient to the pneumatic system via an invasive patient interface 152.

Ventilation may be achieved by invasive or non-invasive means. Invasive ventilation, such as invasive patient interface 152, utilizes a breathing tube, particularly an endotracheal tube (ET tube) or a tracheostomy tube (trach tube), inserted into the patient's trachea in order to deliver air to the lungs. Non-invasive ventilation may utilize a mask or other device placed over the patient's nose and mouth. For the purposes of this disclosure, an invasive patient interface 152 is shown and described, although the reader will understand that the technology described herein is equally applicable to any invasive or non-invasive patient interface.

Airflow is provided via ventilation tubing circuit 130 and invasive patient interface 152. Ventilation tubing circuit 130 may be a dual-limb (shown) for carrying gas to and from the patient 150. In a dual-limb embodiment as shown, a “wye fitting” 170 may be provided to couple the patient interface 154 to an inspiratory limb 132 and an expiratory limb 134 of the ventilation tubing circuit 130.

The tubing circuit may have a bacterial and/or viral filter 180. The filter 180 can be attached at various points on the tubing circuit. In the present example, the filter 180 is located on the expiratory limb.

Pneumatic system 102 may be configured in a variety of ways. In the present example, system 102 includes an expiratory module 110 coupled with the expiratory limb 134 and an inspiratory module 104 coupled with the inspiratory limb 132. Compressor 106 or another source(s) of pressurized gases (e.g., air, oxygen, and/or helium) is coupled with inspiratory module 104 to provide a gas source for ventilatory support via inspiratory limb 132. Reprocessing module 108 is coupled with the expiratory module 110 and compressor 106 to provide reprocessed expired air to the compressor.

The pneumatic system may include a variety of other components, including sources for pressurized air and/or oxygen, mixing modules, valves, sensors, tubing, accumulators, etc. Controller 112 is operatively coupled with pneumatic system 102, signal measurement and acquisition systems, and an operator interface 120 may be provided to enable an operator to interact with the ventilator 100 (e.g., change ventilator settings, select operational modes, view monitored parameters, etc.). Controller 112 may include memory 114, one or more processors 118, storage 116, and/or other components of the type commonly found in command and control computing devices.

The memory 114 is computer-readable storage media that stores software that is executed by the processor 118 and which controls the operation of the ventilator 100. In an embodiment, the memory 114 includes one or more solid-state storage devices such as flash memory chips. In an alternative embodiment, the memory 114 may be mass storage connected to the processor 118 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 118. Computer-readable storage media includes volatile and non-volatile, removable and non-removable media implemented in any method or technology for storage of information such as computer-readable instructions, data structures, program modules or other data. Computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

As described in more detail below, controller 110 issues commands to pneumatic system 102 in order to control the breathing assistance provided to the patient by the ventilator. The specific commands may be based on inputs received from patient 150, pneumatic system 102 and sensors, operator interface 120 and/or other components of the ventilator. In the depicted example, operator interface includes a display 122 that is touch-sensitive, enabling the display to serve both as an input and output device.

As will be described further herein, the controller 110 may be configured to communicate FIO₂ levels and lower threshold levels specified by the clinician to the reprocessing module 108. The controller 110 may also be configured to regulate the oxygen concentration produced by the reprocessing module as well as the duration of use of the molecular sieves used during adsorption by the reprocessing module 108.

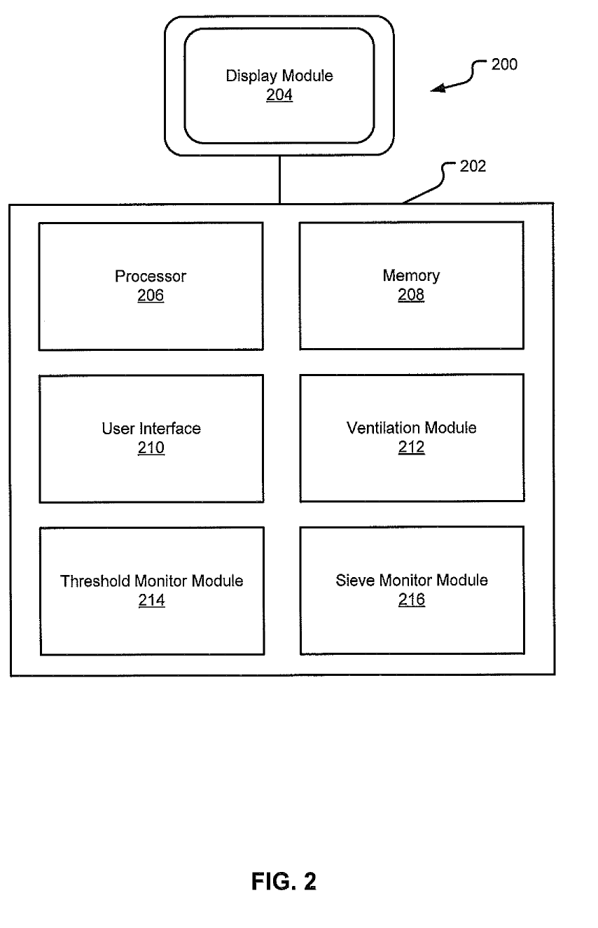

FIG. 2 is a block-diagram illustrating an embodiment of a ventilatory system 200 for oxygen reprocessing of expired gases as described herein.

The ventilator 202 includes a display module 204, memory 208, one or more processors 206, user interface 210, a threshold monitor module and a sieve monitor module. Memory 208 is defined as described above for memory 112. Similarly, the one or more processors 206 are defined as described above for the one or more processors 116. Processors 206 may further be configured with a clock whereby elapsed time may be monitored by the system 200.

The display module 204 displays various input screens to a clinician, including but not limited to an “Oxygen Reprocessing Input Screen,” as will be described further herein, for receiving clinician input. The display module 204 is configured to communicate with user interface 210. Specifically, display module 204 and user interface 210 may receive specified FIO₂ levels and lower threshold level information from a clinician, as described further herein. The user interface 210 may provide various windows and elements to the clinician for input and interface command options. Additionally, user interface 210 may provide useful information to the clinician through display module 204. This useful information may be in the form of various clinical data regarding the patient, displaying for instance, the patient's FiO₂ level, peak inspiratory pressure (PIP), and positive end expiratory pressure (PEEP), etc. Alternately, useful information may be derived by the ventilator 202, based on data gathered from the various ventilation and reprocessing modules 212-226, and the useful information may be displayed in the form of graphs, wave representations, or other suitable forms of display to the clinician. Examples of such graphic representations may include, but are not limited to, pressure and volume curves, flow curves, and pressure-volume loops, etc. Display module 204 may further be an interactive display, whereby the clinician may both receive and communicate information to the ventilator 202, as by a touch-activated display screen. Alternately, user interface 210 may provide other suitable means of communication with the ventilator 202, for instance by a keyboard or other suitable interactive device.

Ventilation module 212 oversees prescribed ventilation as delivered to a patient. Specifically, prescribed ventilation refers to the ventilatory settings for the patient during routine ventilation.

A threshold monitor module 214 may oversee the oxygen concentration of the product gas produced by the reprocessing module 302. Specifically, threshold monitoring module 214 may receive a measurement oxygen concentration of the product gas from threshold testing module 310. The threshold monitoring module can provide the measured oxygen concentration to the display module 204 for display. The threshold monitoring module can also receive a determination by the threshold testing module 310 whether the oxygen content of the product gas is below a lower threshold. If the oxygen concentration is lower than the threshold level, the threshold monitoring module may instruct the display module to set off an “alarm.” An alarm could take the form of a visual alarm, audio alarm, audiovisual alarm, or any other manner of alerting the clinician.

A sieve monitor module 216 may oversee the duration of use of molecular sieves used during the adsorption processes. The sieve monitor module 216 may store the amount of time a particular sieve has been used. The sieve monitor module may also communicate this information to the display module 204 for display.

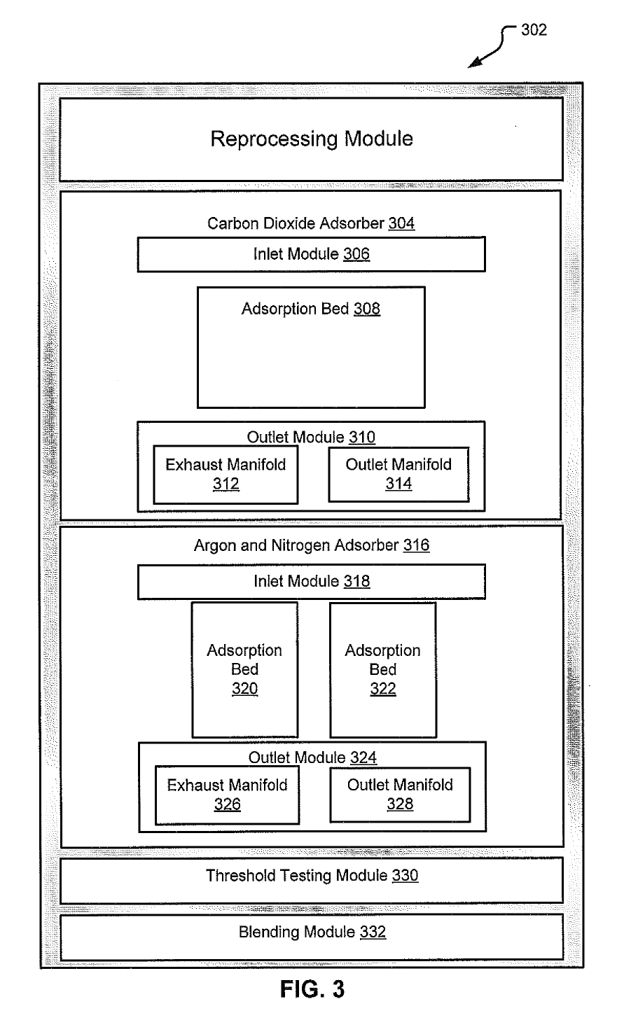

FIG. 3 is an illustration of a reprocessing module for oxygen reprocessing in a ventilation system. Specifically, FIG. 3 illustrates the different elements of the reprocessing module described herein.

The disclosed embodiment of the reprocessing module includes a carbon dioxide adsorber 304. The carbon dioxide adsorber 304 receives filtered exhaled air from the expiratory module 110 at the inlet module 306 of the carbon dioxide adsorber 304. When the filtered exhaled air is received by the inlet module 306, the air is provided to one or more adsorption beds 310. The carbon dioxide adsorber 304 can utilize any type of adsorbent bed and/or molecular sieve, executed singularly or dually, to achieve carbon dioxide adsorption as discussed above. In the present embodiment, only one adsorption bed 310 is used. As discussed above, during the adsorption process, the filtered exhaled air is provided to the adsorption bed 308 and adsorbed using TSA and/or PSA mechanisms. After adsorption, the product gas is received by the outlet manifold 314 of the outlet module 310. The adsorption bed then undergoes desorption in which the adsorbed CO₂ is released into a regeneration gas as described above. The regeneration gas is received by the exhaust manifold 312 of the outlet module 310. The CO₂ laden exhaust air is then exhausted from the reprocessing module 302. The adsorption and desorption processes can be regulated by TSA mechanisms, PSA mechanisms, or other regulation mechanisms. The above described embodiment is but one example of how a carbon dioxide adsorber module that removes carbon dioxide from a gas stream are known in the art and any suitable design, now known or later developed, could be adapted for use in the systems described herein.

The reprocessing module 302 also includes an argon and nitrogen adsorber 316. The argon and nitrogen adsorber 316 receives the product gas from the carbon dioxide adsorber 304 at the inlet module 318. Like the carbon dioxide adsorber 304, the argon and nitrogen adsorber 316 concentrates the oxygen content of the product gas. By means of example and not limitation, the argon and nitrogen adsorber is comprised of dual adsorbent beds 320 and 322. As discussed above, any number of adsorbent beds can be used. By means of example and not limitation, each adsorbent bed contains a molecular sieve. As discussed above, during the adsorption process, the product gas is provided to the molecular sieves and adsorbed using TSA and/or PSA. After adsorption, the product gas is received by the outlet manifold 328 of the outlet module 324. The adsorption bed then undergoes desorption in which the adsorbed molecules are released in a regenerative gas and received by the exhaust manifold 326 of the outlet module 328. The regeneration gas is then exhausted from the reprocessing module 302. The adsorption/desorption cycles of each adsorbent bed 320 and 322 can be executed dually so that while one adsorbent bed 320 is undergoing adsorption the other adsorbent bed 322 is undergoing desorption. The adsorption and desorption processes can be controlled by TSA mechanisms, PSA mechanisms, or other regulation mechanisms. The above described embodiment is but one example of how a argon and nitrogen adsorber module that removes argon and nitrogen from a gas stream are known in the art and any suitable design, now known or later developed, could be adapted for use in the systems described herein.

The reprocessing module 302 also contains a threshold testing module 330. The threshold testing module 330 receives the product gas from the argon and nitrogen adsorber 316. The threshold testing module 330 tests the oxygen concentration of the air output by the carbon dioxide adsorber 304 and argon and nitrogen adsorber 316. The threshold testing module 330 is communicatively coupled to the threshold monitor module 214. The threshold testing module 330 communicates the oxygen concentration of the product gas to the threshold monitor module 214. The threshold testing module 330 is also communicatively coupled to the user interface 210. The threshold testing module 330 receives the lower threshold from the user interface 210. The threshold testing module then determines whether the oxygen content of the product gas is below the lower threshold. The threshold testing module 330 communicates this determination to the threshold monitor module 214. The above described embodiment is but one example of how a threshold testing module that tests oxygen concentration of a gas stream and determines whether the oxygen concentration of a gas stream is below a threshold value are known in the art and any suitable design, now known or later developed, could be adapted for use in the systems described herein.

The reprocessing module 302 also contains a blending module 332. The blending module 332 can contain any number of storage units for room air and/or pure oxygen. The blending module 332 can also connect to exterior sources of room air and/or pure oxygen by any feasible connection mechanism. The blending algorithm module 332 is communicatively coupled to the user interface 210. The blending module 302 receives the product gas from the threshold testing module 330. The blending module 332 mixes the product gas with appropriate contents of room air and/or pure oxygen by applying a blending algorithm reflecting the clinically specified FIO₂ level provided by the user interface 210. The above described embodiment is but one example of how a blending module that blends a gas stream with room air and/or pure oxygen are known in the art and any suitable design, now known or later developed, could be adapted for use in the systems described herein.

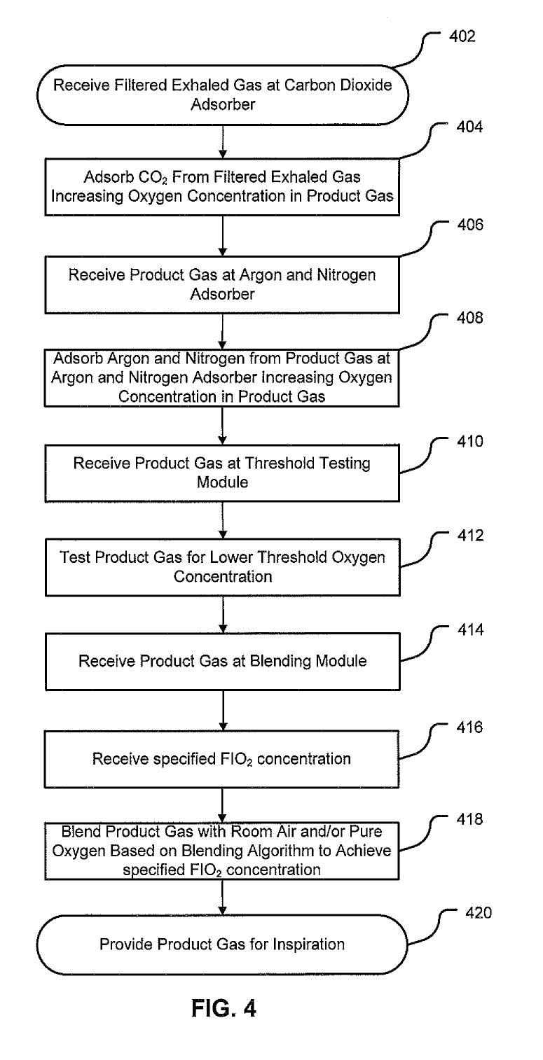

FIG. 4 is a flow-diagram illustrating embodiments of a method for oxygen reprocessing of expiratory gases in ventilation as described herein.

At step 402, the carbon dioxide adsorber 304 receives filtered exhaled air. The carbon dioxide adsorber 304 is configured to selectively adsorb carbon dioxide. The filtered exhaled air could be received from an expiratory module 110, from the expiratory limb 134, or from any other source of expiratory gas used in ventilation.

As step 404, carbon dioxide is adsorbed from the filtered exhaled air increasing the oxygen concentration in the product gas. As discussed above, carbon dioxide adsorption occurs at the carbon dioxide adsorber 304. The carbon dioxide adsorber 304 can contain any number of adsorbent beds. The carbon dioxide adsorber 304 can further utilize any type of adsorbent bed and/or molecular sieve, executed singularly or dually, to achieve carbon dioxide adsorption as discussed above. The filtered exhaled air is received at the adsorbent bed of the carbon dioxide adsorber 304 and carbon dioxide is adsorbed. After the carbon dioxide is adsorbed, the product gas exits the adsorbent bed and the carbon dioxide adsorber 304.

At step 406, the product gas is received at the argon and nitrogen adsorber 316. The argon and nitrogen adsorber 316 is configured to selectively adsorb argon and nitrogen. By means of example and not limitation, the argon and nitrogen adsorber 316 utilizes adsorbent beds with dual molecular sieves.

At step 408, argon and nitrogen are adsorbed from the product gas at the nitrogen argon adsorber 316 increasing the oxygen concentration in the product gas. As discussed above, the present embodiment adsorbs argon and nitrogen by utilizing dual molecular sieves. The product gas is received by a molecular sieve of the dual molecular sieves. The molecular sieve undergoes adsorption of argon and nitrogen wherein argon and nitrogen molecules diffuse through the pores of the one of the dual molecular sieves. At the same time, the other dual molecular sieve undergoes desorption wherein the adhered argon and nitrogen molecules are released from the molecular sieve and exhausted from the argon and nitrogen adsorber 316.

At step 410, the product gas is received at a threshold testing module 330. As discussed above, many methods exist to measure oxygen concentration in a gas. The threshold testing module 330 can utilize one or more than one of the above techniques. The threshold testing module 330 can also be configured to utilize a method not listed above to measure the oxygen concentration in a gas. The threshold testing module 330 is communicatively coupled with the threshold monitoring module 214.

At step 412, the oxygen concentration of the product gas is measured to determine if the oxygen concentration surpasses the lower threshold of oxygen concentration. The testing threshold module 330 compares the oxygen content of the product gas to the lower threshold received from the user interface 210 to determine whether the oxygen content of the product gas is below the lower threshold. The threshold testing module 330 can be further configured to communicate the measured oxygen content and the determination of whether the oxygen concentration of the product gas is below the lower threshold to the threshold monitoring module 214.

At step 414, the product gas is received by the blending module 332. The blending module 332 can contain storage units for room air and/or pure oxygen. The blending module 332 can also be connected to an exterior source of room air and/or pure oxygen. The blending module 332 is communicatively coupled to a user interface 210 that receives a specified FIO₂ level from a clinician.

At step 416, the blending module 332 receives a specified FIO₂ level. The specified FIO₂ level may be greater for patients experiencing difficulty absorbing oxygen into the bloodstream.

At step 418, the product gas is blended with room air and/or pure oxygen based on a blending algorithm. The blending algorithm is formulated to achieve the specified FIO₂ level reflecting the desired oxygen concentration.

At step 420, the product gas is provided for inspiration. Providing for inspiration may include providing the product gas to a compressor 106 or an inspiratory module 104. It may also include providing the product gas directly to the inspiratory limb 132 of the ventilator.

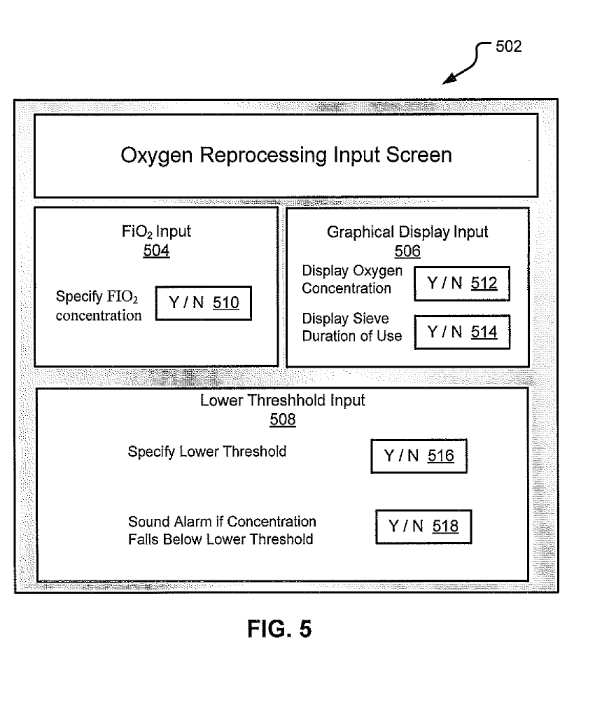

FIG. 5 is an illustration of an embodiment of a graphical user interface for receiving clinician input for oxygen reprocessing of expiratory gases as described herein. Specifically, FIG. 5 illustrates an embodiment of the “Oxygen Reprocessing Input Screen” 502, as described above with reference to display module 204, threshold monitor module 214, and sieve monitor module 216.

The disclosed embodiment of the Oxygen Reprocessing Input Screen 502 displays various input categories, or windows, and entry or command portals, or elements, wherein a clinician may communicate parameters and commands to the ventilator. Disclosed windows and elements may be arranged in any suitable order or configuration such that information may be communicated by the clinician to the ventilator in an efficient and orderly manner. Windows disclosed in the illustrated embodiment of the Oxygen Reprocessing Input Screen 502 may be configured with elements for calling on alternate display input screens or graphical data display screens as may be provided by the ventilator. Disclosed windows and elements are not to be understood as an exclusive array, as any number of similar suitable windows and elements may be displayed for the clinician within the spirit of the present disclosure. Further, the disclosed windows and elements are not to be understood as a necessary array, as any number of the disclosed windows and elements may be appropriately replaced by other suitable windows and elements without departing from the spirit of the present disclosure. The illustrated embodiment of the Oxygen Reprocessing Input Screen 502 is provided as an example of potentially useful windows and elements that may be provided to the clinician to facilitate the input of parameters and commands relevant to the disclosed oxygen reprocessing of expiratory gases as described herein.

A FiO₂ Input window 504 may be provided wherein the clinician can select a desired FiO₂ level for inspiration. Specifically, the clinician can specify FiO₂ level 510. The specified FiO₂ level will be communicated to the blending module 332 and the blending module 332 will create an appropriate blending algorithm based on the specified FiO₂ level.

A Graphical Display Input window 506 may also be provided wherein the clinician can select whether the oxygen concentration and/or sieve duration of use should be graphically displayed. Specifically, the Display Input window 506 receives the oxygen concentration of the product gas from the threshold monitoring module 214 and the sieve duration of use from the sieve monitor module 216. The clinician can decide whether to display the oxygen concentration using the Display Oxygen Concentration button 512. The clinician can also decide whether to display the sieve duration of use using the Display Sieve Duration of Use button 514.

A Lower Threshold Input window 508 may also be provided wherein the clinician can monitor and manage the lower threshold of oxygen concentration in the product gas. Specifically, the clinician can decide whether to specify a lower threshold for oxygen concentration 516. The Lower Threshold Input window 508 communicates the lower threshold to the threshold testing module 330. The Lower Threshold Input window 508 also receives information from the threshold monitoring module 214 about whether the oxygen concentration has fallen below the lower threshold. The Lower Threshold Input window 508 provides the clinician with a button 518 to activate an alarm function that will be set off if the oxygen concentration drops below the lower threshold.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

1. A method executable on a computerized ventilator for oxygen reprocessing of expiratory gases during ventilation of a patient, comprising:
receiving filtered exhaled air at a carbon dioxide adsorber configured to selectively adsorb carbon dioxide;
adsorbing carbon dioxide at the carbon dioxide adsorber from the filtered exhaled air creating product gas with increased oxygen concentration;
receiving the product gas at an argon and nitrogen adsorber configured to selectively adsorb argon and nitrogen from the product gas;
adsorbing argon and nitrogen from the product gas creating product gas with increased oxygen concentration;
receiving the product gas at a threshold testing module, wherein the threshold testing module is configured to measure the oxygen concentration of the product gas;
receiving the product gas at a blending module, wherein the blending module is configured to blend the product gas with room air and/or pure oxygen;
receiving a specified oxygen concentration at the blending module;
blending the product gas with the room air and/or the pure oxygen based on a blending algorithm, wherein the blending algorithm is based on the specified oxygen concentration; and
providing the product gas for inspiration.

2. The method of claim 1, wherein receiving filtered exhaled air at a carbon dioxide adsorber configured to selectively adsorb carbon dioxide, utilizes at least one of an adsorbent bed packed with adsorbent molecules, an adsorbent bed containing a molecular sieve.

3. The method of claim 2, wherein adsorbing carbon dioxide at the carbon dioxide adsorber from the filtered exhaled air is configured to utilize dual molecular sieves.

4. The method of claim 1, wherein receiving the product gas at an argon and nitrogen adsorber configured to selectively adsorb argon and nitrogen from the product gas utilizes at least one of: an adsorbent bed packed with adsorbent molecules, an adsorbent bed containing a molecular sieve.

5. The method of claim 4, wherein adsorbing argon and nitrogen from the product gas is configured to utilize dual molecular sieves.

6. The method of claim 1, wherein the specified oxygen concentration can be specified by a clinician or a default concentration.

7. The method of claim 1, wherein the carbon dioxide adsorber is configured to utilize at least one of: temperature swing adsorption and pressure swing adsorption.

8. The method of claim 1, wherein the argon and nitrogen adsorber is configured to utilize at least one of: temperature swing adsorption and pressure swing adsorption.

9. The method of claim 1, wherein providing the product gas for inspiration can comprise: providing the product gas to a compressor of the ventilator, providing the product gas to an inspiratory module of the ventilator, or providing the product gas to an inspiratory limb of the ventilator

10. A reprocessing module for oxygen reprocessing of expiratory gases during ventilation of a patient, comprising:
a carbon dioxide adsorber configured to selectively adsorb carbon dioxide from filtered exhaled air creating product gas with increased oxygen concentration;
an argon and nitrogen adsorber configured to selectively adsorb argon and nitrogen from the product gas of the carbon dioxide adsorber creating product gas with increased oxygen concentration;
a threshold testing module configured to measure oxygen concentration of the product gas of the argon and nitrogen adsorber;
a blending module configured to blend the product gas with room air and/or pure oxygen based on a specified oxygen concentration and to provide the product gas for inspiration.

11. The carbon dioxide adsorber of claim 10, wherein the carbon dioxide adsorber configured utilizes at least one of: an adsorbent bed packed with adsorbent molecules, an adsorbent bed containing a molecular sieve.

12. The carbon dioxide adsorber of claim 11, wherein the carbon dioxide adsorber is configured to utilize dual molecular sieves.

13. The argon and nitrogen adsorber of claim 10, wherein the argon and nitrogen adsorber utilizes at least one of: an adsorbent bed packed with adsorbent molecules, an adsorbent bed containing a molecular sieve.

14. The argon and nitrogen adsorber of claim 13, wherein the argon and nitrogen adsorber is configured to utilize dual molecular sieves.

15. The blending module of claim 10, wherein the specified oxygen concentration can be specified by a clinician or a default concentration.

16. The carbon dioxide adsorber of claim 10, further configured to utilize at least one of: temperature swing adsorption and pressure swing adsorption.

17. The argon and nitrogen adsorber of claim 10, further configured to utilize at least one of: temperature swing adsorption and pressure swing adsorption.

18. The blending module of claim 10, wherein configured to provide product gas for inspiration can comprise: providing the product gas to a compressor of the ventilator, providing the product gas to an inspiratory module of the ventilator, or providing the product gas to an inspiratory limb of the ventilator.

19. A graphical user interface for configuring oxygen reprocessing of expiratory gases on a ventilator, the ventilator including a computer having a user interface including the graphical user interface for inputting parameters, the graphical user interface comprising:
at least one window associated with the user interface;
one or more selection elements within the at least one window, comprising at least one of:
one or more input elements for enabling the clinician to input parameters for delivering reprocessed oxygen; and
one or more graphic elements for enabling the clinician to generate graphic displays of data collected during the oxygen reprocessing.

20. The graphical user interface of claim 19, wherein at least one of the one or more input elements receives a FIO₂ selection for use when delivering reprocessed oxygen during ventilation.
